(19) United States
(12) Patent Application Publication
(10) Pub. No.: US 2004/0234511 A1
(43) Pub. Date: Nov. 25, 2004

(54) Immune tolerance to predetermined antigens

(75) Inventors: Uri Galili, Chicago, IL (US); Haruko Ogawa, Chicago, IL (US)

Correspondence Address: FOLEY & LARDNER, 150 EAST GILMAN STREET, P.O. BOX 1497, MADISON, WI, 53701-1497, US

(73) Assignee: Rush University Medical Center

(21) Appl. No.: 10/789,955

(22) Filed: Feb. 27, 2004

Related U.S. Application Data
(63) Continuation of application No. PCT/US02/25283, filed on Aug. 9, 2002.
(60) Provisional application No. 60/315,434, filed on Aug. 28, 2001.

(51) Int. Cl. A61K 48/00

(52) U.S. Cl. 424/93.21

(57) ABSTRACT

The present invention provides compositions and methods for inducing immune tolerance to one or more specific antigens in a host mammal. Generally, the methods involves engineering white blood cells, in vitro, to express an antigen which is not native to the host mammal. Cells engineered ex vivo are then introduced into the host mammal to induce immune tolerance to the expressed antigen.

### CLAIM OF PRIORITY

This application claims priority to U.S. Provisional Patent Application No. 60/315,434 filed on Aug. 28, 2001, and is a continuation-in-part of International Application No. PCT/US02/025283 filed on Aug. 9, 2002, the entire contents of both of which are hereby incorporated by reference.

### STATEMENT REGARDING FEDERALLY SPONSORED RESEARCH

This invention was made with Government support awarded by NIH Grant No. AI45849. The Government has certain rights in this invention.

### TECHNICAL FIELD

The present invention relates to the induction of immune tolerance to a predetermined antigen such as a carbohydrate antigen, or a protein antigen and more specifically to the induction of immune tolerance by the expression of that predetermined antigen on autologous white blood cells.

### BACKGROUND OF THE INVENTION

Red cells and other cells in human populations express carbohydrate antigens such as blood group A [GalNAcα1-3(Fucα1-2)Galβ1-(3)4GlcNAc-R] or B antigens [Galα1-3(Fucα1-2)Galβ1-(3)4GlcNAc-R]. Individuals that lack these antigens have antibodies against the corresponding antigen i.e. blood group A individuals have anti-B antibodies, blood group B individuals have anti-A antibodies and blood group O individuals have anti-A and anti-B antibodies. Such antibodies prevent transplantation of ABO incompatible organs. For example, transplantation of an organ such as kidney from a blood group A donor to a blood group B recipient results in the rejection of the graft by the pre-existing natural anti-A antibodies in the recipient and by elicited anti-A antibodies produced by the recipient's immune system against blood group A antigen on the allograft. Accordingly, blood group A recipient will reject the kidney from a blood group B donor and blood group O individual will reject the kidney from either a blood group A donor or blood group B donor.

A similar mechanism mediates the rejection of xenografts (e.g. pig kidney or pig heart) in humans. This rejection occurs because all humans produce the natural anti-Gal antibody which constitutes ˜1% of circulating immunoglobulins and which binds specifically to the α-gal epitope [Galα1-3Galβ1-(3)4GlcNAc-R], abundantly expressed on pig cells and other nonprimate mammalian cells [Galili Immunology Today 1993]. Xenograft recipients also produce large amounts of elicited anti-Gal antibodies as part of the immune response to the α-gal epitopes on the xenograft. The binding of the human natural anti-Gal and of the elicited anti-Gal to this carbohydrate epitope expressed on cells of the graft, results in effective rejection of the xenografts.

Removal of these anti-carbohydrate antibodies prior to transplantation does not prevent ABO mismatched allograft rejection, or xenograft rejection, because the immune system continues to produce high affinity IgG antibodies against these carbohydrate antigens, causing the rejection of allografts or xenografts. Therefore, induction of immune tolerance to these carbohydrate antigens will be beneficial in the prevention of the rejection of ABO incompatible (mismatched) allografts, or of xenografts.

In addition to the immune response to incompatible carbohydrate antigens, the immune system reacts against peptide antigens such as MHC (major histocompatibility complex allo-antigens) on allografts and against peptide xenoantigens on xenografts. Whereas, xenotransplantation is a practice of the future, the extensive immune response to ABO incompatible allografts exacerbates their rejection, to the extent that in the USA, transplantation of such grafts from ABO incompatible donors (e.g. kidney donation between close relatives) is usually not practiced. It is practiced, however, in Europe and Japan, where the incompatible anti-blood group antibodies and the spleen of the recipient are usually removed prior to transplantation. The induction of tolerance to the carbohydrate antigens is likely to reduce the overall immune response to the graft, because of the lack of anti-blood group immune response, and is likely to obviate the need for removal of the spleen in the recipient. Induction of tolerance to protein antigens such as major histocompatibility complex (MHC) antigens is likely to decrease the tendency of graft recipients to reject their graft.

### SUMMARY OF THE INVENTION

The present invention provides methods and composition for suppressing an immune response of a mammal to a desired antigen. The present methods can also be used to induce partial or complete immune tolerance of a desired antigen in a mammal. As used herein, immune tolerance includes, but does not require, providing complete tolerance against an antigen of interest in an animal. Generally, the antigens to which immune tolerance induced are not native, i.e. not naturally produced, by the mammal.

Generally, the present methods involve engineering white blood cells to express at least a portion of an antigen of interest. According to these methods, a population of white blood cells, which can be present in a cell population made up primarily of white blood cells or can be white blood cells in a mixed cell population, are engineered to express at least a portion of an antigen of interest. Preferably the portion of the antigen of interest is itself, antigenic. The portion of the antigen of interest can also be the entire antigen where desired. Different cells in the white blood cell population can be engineered to express the same or different portions of the antigen of interest.

The engineered white blood cells expressing the at least a portion of the antigen of interest are then administered to an individual of interest thereby inducing immune tolerance in the individual to the antigen of interest. The present methods can further involve obtaining and/or isolating a white blood cell population from a mammal, and in particular the mammal of interest. Once obtained the white blood cell population can be expanded to provide an additional source of the white blood cells. In some embodiments, the white blood cells engineered to express at least a portion of the antigen of interest are from an individual other than the individual to whom the engineered white blood cells are administered. In preferred embodiments, the white blood cells are obtained from, and administered back into, the same individual or patient.

The present invention also provides compositions containing the white blood cells engineered to express the antigen of interest. In particular, pharmaceutical compositions containing the engineered white blood cells for administration to the mammal or patient are provided.

The present invention can also provide an animal model for inducing immune tolerance to a desired antigen or antigens. According to these embodiments, white blood cells expressing a specific antigen are administered to an animal and the animal is then subjected to the antigen, such as through tissue transplantation, antigen injection, via autologous tissue in the case of an autoimmune disease or the like. The response of the animal to the antigenic stimuli can then be measured. These models can be used to measure the antigenicity of a specific antigen and/or the effectiveness of the present compositions and techniques in inducing immune tolerance to the antigens. The response of the animal to the antigenic stimuli can also be compared to the response of a control animal which has not received the engineered white blood cells.

In other embodiments, mature B lymphocytes, capable of producing antibodies to cell surface antigens, such as carbohydrate antigens, are induced to undergo immune tolerance when they encounter the cognate antigen expressed on autologous white blood cells such as lymphocytes and monocytes. The basis for that tolerance is believed to be that in the absence of any T cell help, the cross linking of B cell receptors by the cognate carbohydrate antigen on autologous cells results in tolerance induction on the B cell. Antigens such as blood group A or B antigens, or the α-gal epitope, do not activate T cells, because their interaction with T cell receptors can not include the accessory molecules of the receptor. This type of tolerance can be induced by using autologous white blood cells, in particular peripheral blood lymphocytes, engineered to express the carbohydrate antigen. One method for achieving expression of antigens, such as carbohydrate antigens, is by transduction of lymphocytes and other white blood cells with a replication defective adenovirus vector that contains the gene encoding for the predetermined antigen. In the case of carbohydrate antigens the gene inserted into the adenovirus genome is the glycosyltransferase gene encoding the enzyme that synthesizes the carbohydrate antigen. Such genes can also be introduced into the white blood cells by any suitable method that introduces genes into cells, such as electroporation of naked DNA plasmids. The transduced cells are administered into the mammalian host subsequent to the removal of circulating antibodies against the antigen. B cells encountering the autologous transduced cells expressing the antigen will undergo tolerance.

In yet another embodiment, the white blood cells are transduced with a gene encoding for a protein such as MHC molecules and administered back to the mammalian host. Prevention of T cell response to that protein antigen is achieved by any clinically acceptable method for T cell immunosuppression. B cell exposure to that protein antigen in the absence of T cell help results in tolerance of these B cells.

### BRIEF DESCRIPTION OF THE DRAWINGS

FIG. 1. Mechanisms for activation and tolerance of anti-Gal B cells.

A. In xenograft recipients, xenoglycoproteins expressing α-gal epitopes contain multiple immunogenic xenopeptides. The xenoglycoproteins are internalized by the anti-Gal B cell, subsequent to interaction of α-gal epitopes with anti-Gal B cell receptors (BCR). The processed immunogenic xenopeptides (●, ▪, ▴) are presented in association with class II MHC molecules and effectively activate many helper T cells with the corresponding T cell receptors (TCR) specificities. These activated T cells provide the help to the B cell to complete its activation, undergo proliferation, isotype switch and affinity maturation, for the ultimate production of high affinity anti-Gal IgG.

B. Tolerance induction on naive and memory anti-Gal B cells as a result of anti-Gal B BCR binding α-gal epitopes on autologous or syngeneic cells. This interaction leads to clustering of the BCR resulting in tolerization signal.

FIG. 2. Expression of α-gal epitopes on HeLa cells transduced by AdαGT (1×10¹⁰ MOI/ml), as measured by flow cytometry of cells stained with BS lectin (A) of with human anti-Gal (B). Closed histograms—untransduced cells; open histograms—cells transduced with AdαGT.

FIG. 3. In vivo follow up of KO lymphocytes that were transduced in vitro with AdαGT and administered into irradiated KO mice. KO lymphocytes were transduced for 4 h by 10¹⁰ infectious units/ml of AdαGT, washed and administered into irradiated KO recipients. (a) KO lymphocytes transduced with parental adenovirus vector lacking α1,3GT gene, obtained on day 1 post transduction.; (b) AdαGT transduced lymphocytes obtained on day 1; (c) day 2; (d) day 3; (e) day 4; (f) day 7; (g) KO lymphocytes; (h) WT lymphocytes. Lymphocytes were stained by FITC-BS lectin (10 μg/ml) and assayed by flow cytometry. Note that AdαGT transduced lymphocytes expressed α-gal epitopes on days 1-3, but not on day 7.

FIG. 4. PCR analysis of lymphocytes for intact exon 9 of α1,3GT gene. Genomic DNA was extracted from spleen lymphocytes and subjected to PCR for the exon 9 of the mouse α1,3GT gene. (a) PCR analysis for WT lymphocytes using 3-200 ng template DNA. (b) PCR analysis for KO lymphocytes transduced with AdαGT, 24 h post transduction, using template DNA as for WT lymphocytes. (c) PCR analysis for KO lymphocytes obtained 1, 2, 3, 4 and 7 days post transduction, using 500 ng template DNA. (d) PCR analysis for KO lymphocytes from five tolerized mice, which were transferred into five secondary recipients. The PCR reaction included 500 ng DNA as template and all were negative for intact exon 9.

FIG. 5. Expression of α-gal epitopes on AdαGT transduced lymphoid populations. Splenocytes were double stained with BS lectin and anti-mouse CD3 (a-c), anti-mouse CD45R/B220 (d-f) or anti-mouse CD11b/Mac-1 (g-i), 2 days posttransduction. (a, d, g) KO lymphoid cells transduced with control empty adenovirus vector; (b, e, h) KO lymphoid cells transduced with AdαGT; (c, f, i) WT lymphoid cells. Proportion (%) of double-stained cells in each population is indicated. The low binding of BS lectin to KO cells transduced with parental adenovirus vector is the nonspecific background level.

FIG. 6. Tolerance induction on naive anti-Gal B cells by AdαGT transduced lymphocytes. (a) Production of anti-Gal IgG in tolerized mice receiving AdαGT transduced lymphocytes and immunized four times with PKM. Anti-Gal response in 10 KO mice receiving AdαGT transduced lymphocytes (∘) or in 10 control mice receiving lymphocytes transduced with parental adenovirus vector (●). (b) Mean±s.e. of anti-Gal IgG response in 10 KO mice tolerized with AdαGT (∘) or in 10 control mice (●). Statistical analysis by Student's t-test indicated significant differences between the two groups at all serum dilutions (P<0.001). (c) Production of anti-non-Gal IgG in the KO mice presented in FIG. 6a as measured by ELISA with wells coated with PKM. Anti-non-Gal activity in control KO mice (●) or in tolerized mice (∘). Representative data of three out of 10 mice in each group with similar results.

FIG. 7. Tolerance induction on memory anti-Gal B cells by AdαGT transduced lymphocytes. (a) Production of anti-Gal in tolerized mice receiving AdaGT transduced lymphocytes and immunized with PKM, 2 and 3 weeks post adoptive transfer. Anti-Gal IgG response in 16 KO mice tolerized by AdαGT transduced lymphocytes and producing anti-Gal at a level of <1.0 OD at serum dilution of 1:50 (∘), in 12 KO mice tolerized by AdαGT transduced lymphocytes and producing anti-Gal at a level of >1.0 OD at serum dilution of 1:50 (Δ), or in 18 control mice receiving lymphocytes transduced with parental ‘empty’ adenovirus vector (●). (b) Mean±s.e. of anti-Gal response in the three groups of KO mice presented in FIG. 7a. Statistical analysis by Student's t-test indicated significant differences between the control and both of tolerized groups at all serum dilutions (P<0.00001). (c) Production of anti-Gal IgM in the group of control mice (●) or in mice that were effectively tolerized by AdαGT transduced KO lymphocytes (∘), as in FIG. 7a. Data are presented as mean±s.e. of 15 mice in each group. Statistical analysis by Student's t-test indicated significant differences between the two groups at all serum dilutions (P<0.00001).

FIG. 8. Perpetuation of tolerance by WT heart. Anti-Gal IgG response in four irradiated KO mice that received memory anti-Gal B cells, tolerized, transplanted with WT heart and immunized four times with PKM, as described in the timeline protocol in Table 1 (∘). The antibody activity was measured in the serum 1 week after the fourth PKM immunization. Anti-Gal response in control mice receiving the same primed anti-Gal B cells and immunized twice with PKM (●).

FIG. 9. Analysis of anti-Gal production after secondary transfer of lymphocytes from tolerized mice. (a) Lymphocytes were transferred from tolerized mice or control mice (presented in FIG. 7a) into irradiated secondary recipients (20×10⁶ lymphocytes per mouse). These recipients (n=5 in each group) were immunized twice with PKM, starting 14 days post second adoptive transfer. Anti-Gal response in secondary recipients of tolerized lymphocytes (∘) or of control lymphocyes (●) was measured 1 week after the second PKM immunization. (b) Analysis of regulatory cell activity in tolerized mice by evaluating anti-Gal IgG response after secondary transfer of 20×10⁶ lymphocytes from control mice (●), or of mixed populations of 20×10⁶ lymphocytes from control mice and 20×10⁶ lymphocytes from tolerized mice (∘). The secondary recipients (n=5 in each group) were immunized twice with PKM starting 14 days post adoptive transfer.

### DETAILED DESCRIPTION OF THE INVENTION

The present invention relates to the induction of immune tolerance to a predetermined antigen (or immunogen) that here is illustratively a carbohydrate antigen. In accordance with another embodiment, a host mammal substantially free of circulating antibodies that specifically immunoreact with the antigen is provided. Autologous white blood cells (from the host mammal) that express the predetermined antigen on the cell surface are administered (inoculated) into the blood stream of the host mammal one or more times. The host mammal can be a human patient or non-human mammal, such as a mouse, rat, dog, cat, horse, cow, goat, pig or the like.

The studies disclosed herein indicate that autologous white blood cells from a patient that are transduced in vitro with a glycosyltransferase gene within a virus or other vector, when inoculated back into the patient, induced immune tolerance to the carbohydrate epitope produced on the transduced cells after expression of the glycosyltransferase. The subsequent transplantation of a graft expressing the carbohydrate epitope does not induce antibody production against that epitope. Without limiting the scope of the invention, it is believed that tolerance is induced primarily by lymphocytes expressing the tolerizing antigen. These lymphocytes are capable of effectively migrating to the lymphoid organs where they encounter the B cells which are the target for tolerance induction.

The present methods preferably use cell populations that are primarily composed of white blood cells. More preferably the white blood cells will make up greater than 90 percent, such as 95, 99 percent or more, of the cells in the population, and in particular lymphocytes. One disclosed method for obtaining a cell sample enriched in white blood cells is disclosed in U.S. Pat. No. 5,785,869. In some preferred embodiments the white blood cells are isolated from a cell population obtained from a patient or patients of interest, such as in individual having an allergic reaction or one which has, or is proposed to undergo, tissue transplantation.

The present disclosure more particularly describes a method for preventing antibody production in host mammals by inducing tolerance in that host mammal to a blood group antigen, such as blood group A [GalNAcα1-3(Fucα1-2)Gal-R] or blood group B antigens [Galα1-3(Fucα1-2)Gal-R] in ABO incompatible (mismatched) allograft recipients, or to α-gal epitopes [Galα1-3Galβ1-(3)4GlcNAc-R] in xenograft recipients, where R is the remainder of carbohydrate chain, glycolipid, glycopeptide, glycoprotein or any other molecule to which the antigen is bonded. Such tolerance induction helps in prevention of immune rejection of such grafts.

More specifically, this principle to tolerance induction to carbohydrate antigens was demonstrated in α1,3galactosyltransferase (α1,3GT) knockout mice with splenocytes transduced by adenovirus vector containing the α1,3GT gene. The α1,3GT enzyme adds an alpha-linked (α-linked) galactose to a precursor glycoprotein, glycopeptide, glycolipid or other glycan molecules so that predetermined antigen contains an α-linked galactose (or galactosyl group). Following such treatment, these mice lack the ability to produce the anti-Gal antibody upon immunization with pig kidney membranes expressing α-gal epitopes. This method for tolerance induction can be used in humans for prevention of anti-blood group antibodies in ABO incompatible allograft recipients, or in prevention of anti-Gal production in xenograft recipients.

The method of tolerance induction to a given antigen by in vitro transduction of autologous cells with one or more genes that cause the expression of the antigen followed by administration of the autologous cells expressing the antigen into a mammalian host such as a patient, can be used for inducing tolerance to a variety of antigens such as carbohydrate antigens, MHC antigens, antigens in autoimmune diseases, including those mediated by antibodies (e.g., Graves disease or pernicious anemia), or antigens that cause allergic reactions, such as food antigens or latex antigens. Common food antigens include nut (e.g., peanut, walnut, almond, pecan, cashew, hazelnut, pistachio, pine nut, brazil nut) antigens, such as Ara h 1-3, fish antigens, shellfish (e.g., shrimp, crab, lobster, clams) antigens, such as tropomyosin, egg antigens, cow milk antigens, such as casein lactalbumin, lactoglobulin, bovine albumin, and gamma globulin, seed (e.g., sesame, poppy, mustard) antigens, and wheat antigens, for example glutens, such as prolamines, which include gliadin, and glutelins. Suitable antigens that can be targeted according to the present methods are also discussed in U.S. Patent Application Publication 2004/0009906.

This method of tolerance induction can be used for a number of types of transplantation, autoimmunity and allergy including without limitation those discussed below. In some instances, particularly where a subject is being tolerized for a protein antigen, the T cells of the patient can be suppressed through means known in the art to prevent T cell help. Without limiting the scope of the invention, T cell suppression is believed to be beneficial in order to prevent the T cells from rescuing B cells. Generally, the T cells can be suppressed prior to administration of the transduced cells, during administration of the transduced cells and/or for a period of time after the transduced cells have been administered. Typically, subject immunosuppression will be started at least two to three weeks prior to administration of the transduced cells and discontinued after about two weeks post administration. In some embodiments, such as when the mammal is immunosuppressed, then no additional immunosuppression may be required to induce tolerance to the antigen.

Any suitable immune suppression therapy can be used, such as radiation treatment, administering immunosuppressive drugs, and/or monoclonal antibodies that specifically reduce the activity of T lymphocytes. Immunosuppressive drugs include steroids, such as corticosteroids including prednisone, cyclosporin, tacrolimus (FK506), sirolimus (rapamycin), methotrexate, mycophenolic acid (mycophenolate mofetil), everolimus, azathiprine, and NOX-100. Suitable suppression techniques, which can also include the administration of antiproliferative agents, are discussed in U.S. Patent Application Publication 2004/0009906.

A. Transplantation of ABO Mismatched Allograft:

Blood group A patient that is to receive an allograft from a blood group B donor can exhibit tolerance to blood group B antigen by in vitro transduction of his/her white blood cells, in particular lymphocytes, with an adenovirus vector containing the blood group B transferase [Yamamoto et al., Nature 345:229, 1990], or other suitable vector containing this gene. The transduced cells upon administration into the blood circulation of the patient express blood group B antigen on the white blood cells that induce tolerance to this antigen and thus prevent the production of anti-blood group B antibodies in the graft recipient. The tolerance can be induced because these white blood cells do not activate T cells. In the absence of T cell activation, the interaction between B cells and the blood group B antigens they recognize which is expressed on the white blood cells, results in elimination of these B cells and tolerance induction to the blood group B antigen.

The same tolerance will occur in blood group O recipients of an allograft from a blood group B donor. Thus, tolerance to blood group A antigen can be induced in blood group B or O individuals receiving autologous white blood cells that were transduced with a vector containing blood group A transferase [Yamamoto et al., Nature 345:229, 1990]. This tolerance induction is performed after removal of the corresponding anti-A or anti-B antibody from the blood by columns expressing the corresponding carbohydrate antigen [Bensinger et al., N Engl. J. Med. 304:160, 1981], or by plasmapheresis.

B. Transplantation of Human Patients with a Xenograft:

White blood cells from a patient in need for a xenograft can be transduced in vitro with a vector containing α1,3GT gene (e.g. adenovirus containing this gene and referred to as AdαGT), then administered (inoculated) into the blood circulation of the patient. This results in induction of tolerance to the α-gal epitope and prevent anti-Gal response to α-gal epitopes on the xenograft cells and prevent anti-Gal response upon transplantation of the xenograft. This tolerance induction is performed after removal of anti-Gal from the blood by a column expressing α-gal epitopes [Galili Seminars in Immunopathol. 15:155, 1993], or by plasmapheresis.

The method for induction of tolerance by expression of an antigen on autologous white blood cells, can be used for induction of tolerance to other antigens that can be expressed by in vitro transduction of the autologous cells with the corresponding gene coding for the antigen, or for an enzyme(s) producing the antigen, as indicated in the following examples.

C. Tolerance Induction to MHC Antigens

White blood cells from a patient in need for a graft, such as a kidney graft, and receiving such a graft from a living donor relative can be used to tolerize the patient to the known MHC allo-antigens of the donor. The white blood cells are transduced in vitro, such as with an adenovirus vector containing the gene encoding for the donor's allo-antigens. The transduced white blood cells are administered back into the patient who is simultaneously treated with an immunosuppressive regimen to prevent T cell help. The B cells interacting with the autologous white blood cells that express the transduced MHC antigens will be deleted or ablated. The subsequent transplantation of the organ will result in perpetuation of the state of tolerance since, in the absence of antigen specific B cells, the MHC allo-antigen on the graft will be “regarded” by the immune system of the recipient as a self antigen.

D. Tolerance Induction to Antigens in Autoimmune Diseases

An example of B cell tolerance to an antigen which has a role in autoimmunity is Graves Disease, where antibodies produced by B cells with specificity to thyroid stimulating hormone receptor (TSHR) continuously stimulate the thyroid cells expressing this receptor. According to the present invention, autologous white blood cells including lymphocytes from Graves Disease patients will be transduced in vitro, such as with an adenovirus vector containing the previously cloned human TSHR gene [Misrahi et al. Biochem. Biophys. Res. Comm. 166:349-403, 1990], or this gene will be introduced by any other method to these cells. The transduced white blood cells will be administered back into the patients subsequent to the removal of the anti-TSHR antibodies by various methods such as plasmapheresis. The patient will be also immunosuppressed to prevent T cell help. The administered white blood cells will induce tolerance by deletion of the B cells that are capable of producing anti-TSHR antibodies. Subsequently, the TSHR on the thyroid cells will be regarded as a self antigen that does not elicit an immune response.

E. Tolerance Induction to an Allergen

In individuals who produce allergy mediated antibodies (i.e. IgE antibodies) to a known allergen (an allergy inducing antigen), the production of the antibodies can be prevented by in vitro manipulation of autologous white blood cells that include lymphocytes for expression of the allergen. Subsequently, the autologous white blood cells are administered back into the patient, in whom the circulating antibodies have been removed, such as by plasmapheresis or by any other method, and T cell help is prevented by an immunosuppressive regimen. When they encounter B cells capable of producing the allergy inducing antibodies, the white blood cells expressing the allergen induce the deletion of these B cells.

The experimental model used for understanding tolerance induction to α-gal epitopes is α1,3galactosyltransferase knockout mice (designated KO mice). These mice lack α-gal epitopes and can produce high affinity anti-Gal IgG when immunized with pig kidney membranes (PKM) expressing α-gal epitopes [Tanemura et al., J. Clin. Invest. 105: 301, 2000]. It was found that in order to produce anti-Gal IgG, the anti-Gal producing B cells (designated anti-Gal B cells) need the help of T helper (T_{H}) cells that are activated by many different xenopeptides processed and presented by these B cells.

However, T_{H} cells can not be activated by the α-gal epitope itself. MHC molecules on antigen presenting cells (APCs) can not present cell surface carbohydrate antigens to T cells. This is because most cell surface N-linked (asparagine linked) carbohydrate chains have a size that is similar to the size of a 25-30 amino acid peptide, and they protrude from the groove of MHC molecule on APCs to a considerable distance [Spier et al., Immunity 10:51, 1999]. This protrusion prevents interaction of accessory T cell receptor (TCR) molecules with the corresponding ligands on APCs, after the initial engagement of the TCR with processed carbohydrate antigens.

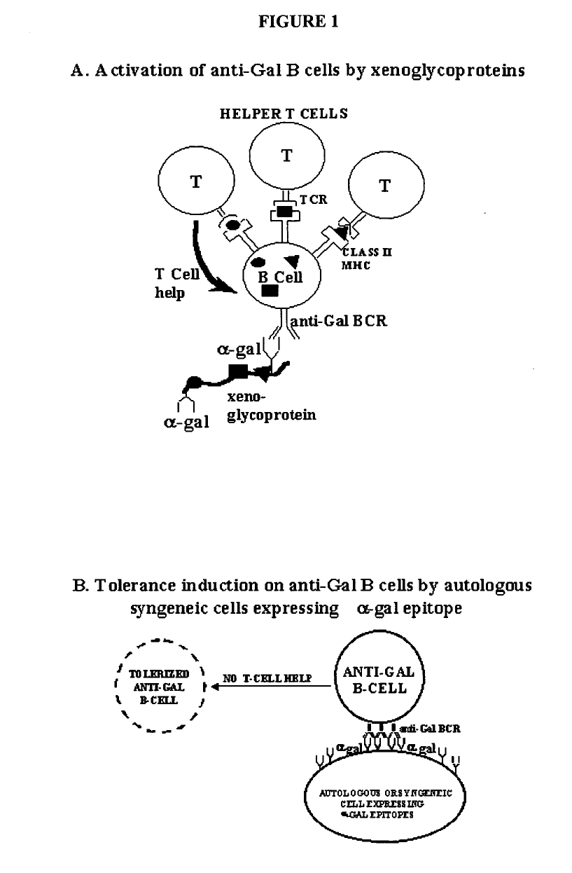

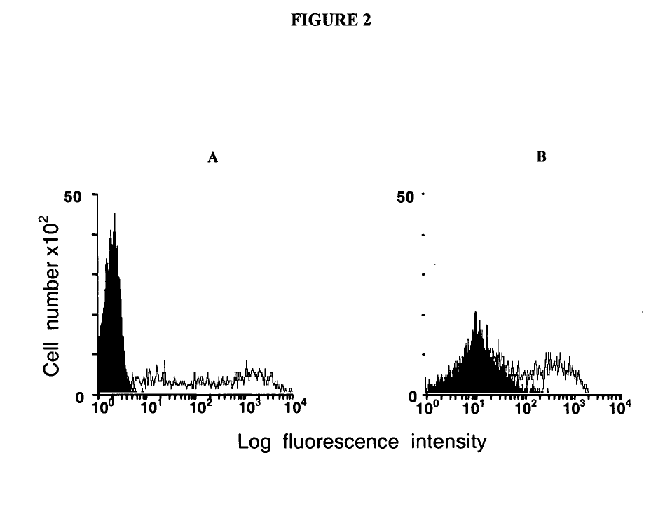

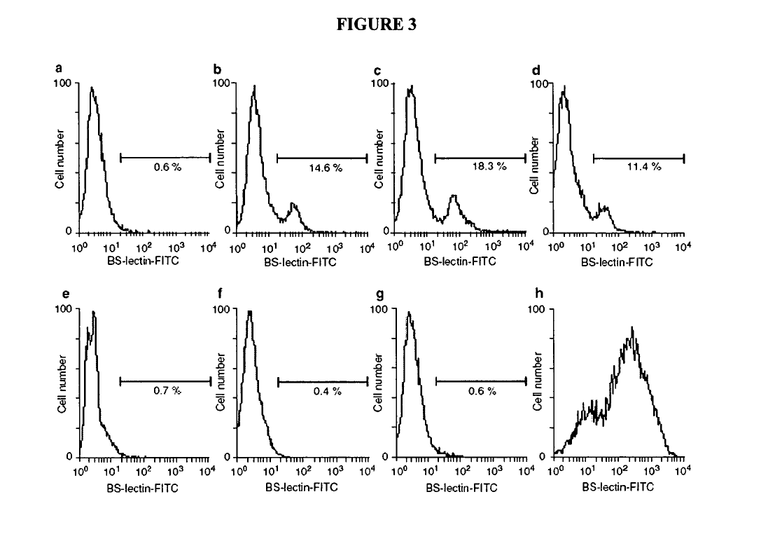

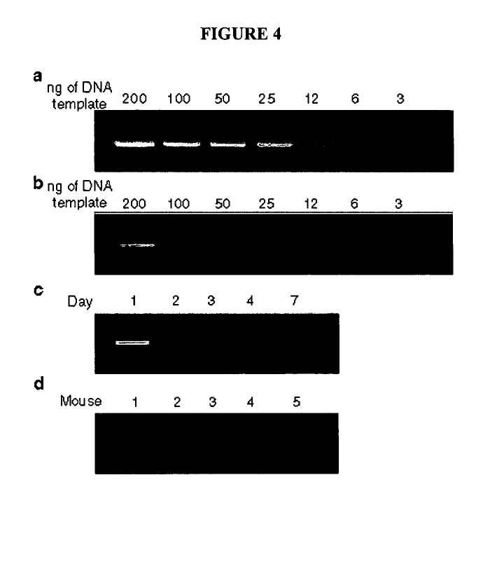

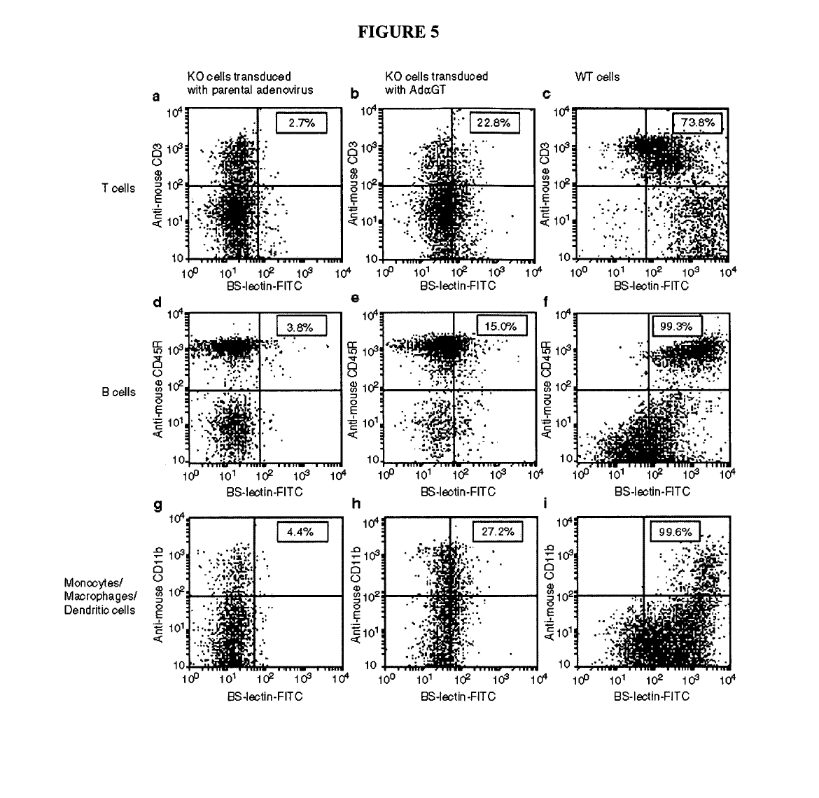

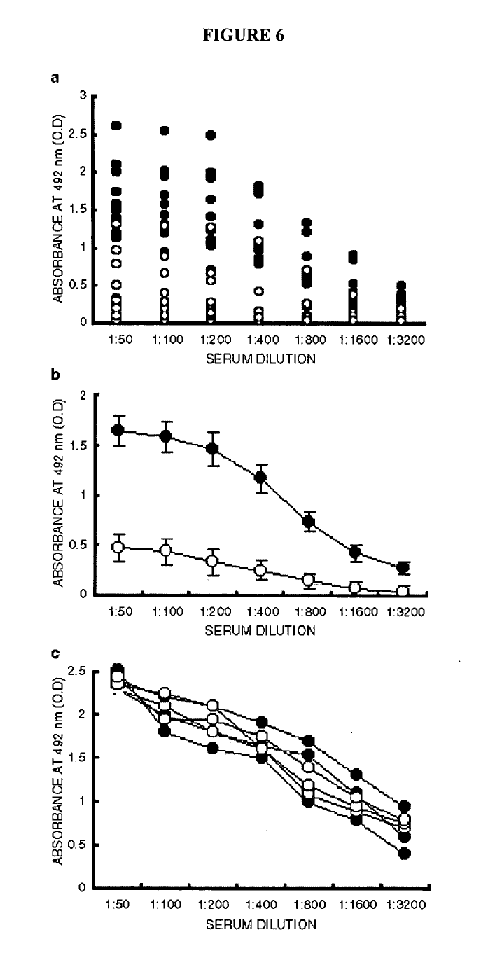

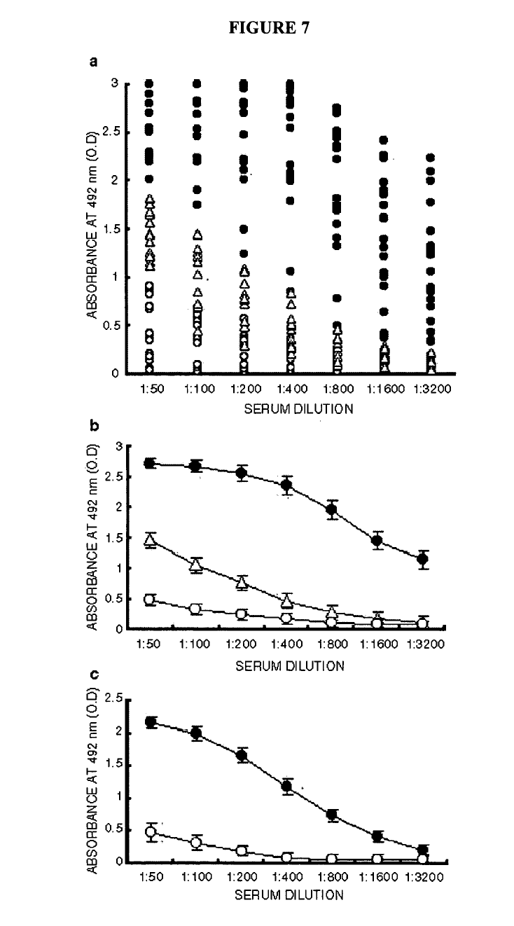

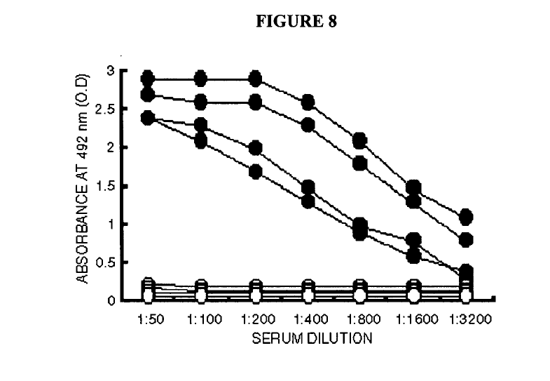

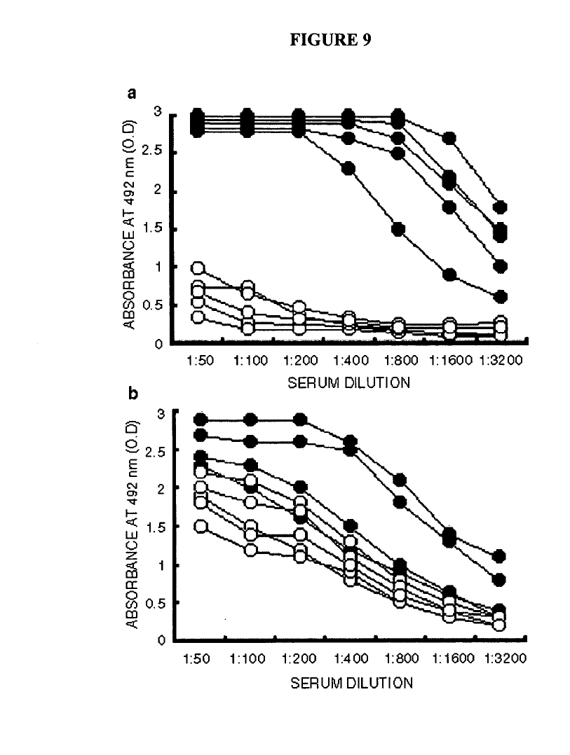

Because of these structural constraints, T cells cannot be activated by carbohydrate chains linked to peptides that are processed and presented by APCs. The activation of the T_{H} cells is enabled, however, by the interaction of the TCR on the T_{H} cells with xenopeptides that are processed and presented by anti-Gal B cells. These xenopeptides originate from xenoglycoproteins released from the xenograft, which engage the B cell receptors on anti-Gal B cells via α-gal epitopes. The glycoproteins are internalized, processed and expressed on these B cells as xenopeptides in association with MHC molecules. As shown schematically in FIG. 1A the interaction of these xenopeptides with the corresponding TCR results in the activation of the T_{H} cells which, in turn, help the anti-Gal B cells to undergo activation for effective production of anti-Gal IgG epitopes [Tanemura et al., J. Clin. Invest. 105: 301, 2000].

Without wishing to be bound by theory, it is believed that in the absence of T_{H} cell help, α-gal epitopes on syngeneic cells (i.e. cells from other animals of the same strain), or autologous cells, rather than on xenogeneic cells, will bind to anti-Gal B cells and induce tolerance by cross linking of the B cell receptors (FIG. 1B). Under such conditions, T_{H} cells are not activated since syngeneic or autologous cells manipulated to express the α-gal epitope, display no antigens that can activate T cells. In the case of protein antigens like MHC allo-antigens, T cell help is prevented by immunosuppression of the T cells by clinically acceptable regimens. It is further believed that the expression of α-gal epitopes on syngeneic or autologous lymphocytes, and other cells, can be achieved by introducing the α1,3GT gene into these cells, by transduction with a replication defective adenovirus vector containing the α1,3GT gene. Administration (inoculation) of autologous white blood cells that were transduced in vitro to express α-gal epitopes is believed to induce tolerance to this epitope.

To prepare a replication defective adenovirus vector containing the α1,3GT gene, the open reading frame of mouse α1,3GT cDNA [Larsen et al., Proc Natl Acad Sci USA 86:8227, 1989] was cloned into human adenovirus type 5. This virus is replication defective because the genes coding for early antigens E1 and E3 were deleted from the virus genome [Gao et al., J. Virol. 70:8934, 1996]. This vector can be propagated as a replicating virus only in the human cell line 293, in which the viral E1 gene is integrated as complementing genes [Gao et al., J. Virol. 70:8934, 1996].

The α1,3GT cDNA was inserted into the adenovirus shuttle plasmid pAd, which then was co-transfected into the 293 cells with the adenovirus vector containing deletions in E1 and E3 regions. The α1,3cDNA was inserted in low frequency into the virus genome by homologous recombination of the flanking regions of the pAd plasmid. The individual plaques containing virus with inserted α1,3GT cDNA were screened by the de novo expression of α-gal epitopes in the human 293 cells. This was measured by the binding of labeled Bandeiraea (Griffonia) simplicifolia IB4 lectin (BS lectin) that interacts specifically with α-gal epitopes on mammalian cells [Wood et al., Arch Biochem Biophys. 198:1, 1979]. The isolated clone of adenovirus containing the α1,3GT gene was designated AdαGT [Deriy et al., Glycobiology 12:135, 2002]. That adenovirus was prepared in supernatants of transduced 293 cell cultures at a concentration of 1×10¹⁰ infectious units (IU)/ml (i.e. viral vector suspension kills 293 cells up to a dilution of 1×10⁻¹⁰).

The ability of AdαGT to induce expression of α-gal epitopes in human HeLa cells which lack α-gal epitopes was described in detail in a recent publication [Deriy et al., Glycobiology 12:135, 2002]. FIG. 2 from that study demonstrates the expression of α-gal epitopes by binding of BS lectin (FIG. 2A) and binding of labeled isolated human anti-Gal (FIG. 2B) [Galili et al., J. Exp. Med. 162:573, 1985]. HeLa cells were incubated with the tissue culture supernatants containing the virus for 4 h at 37° C., then washed and incubated for additional 24 h in culture medium. Subsequently, the cells were found to express many α-gal epitopes as indicated by the binding of BS lectin. By using the ELISA inhibition assay for quantifying α-gal epitopes [Galili et al., Transplantation 65: 1129, 1998], the transduced HeLa cells were found to express on average 2×10⁶ α-gal epitopes per cell 24 h post transduction. Analysis of activity of α1,3GT in the transduced HeLa cells revealed the appearance of α1,3GT in the cells within 6 h post transduction, whereas α-gal epitopes appeared on the cell membrane within 12 h post transduction [Deriy et al., Glycobiology 12:135, 2002].

Any technique for the introduction of heterologous, nucleic acids encoding the antigens or enzymes that produce the antigen into host cells into white blood cells, and particularly lymphocytes, can be adapted to the practice of this invention. In alternative embodiments, the white blood cells of an individual can be engineered in vivo to express the antigen of interest.

The present invention also provides various compositions, which generally include the vectors, white blood cells or progenitor cells described herein. A person having ordinary skill in this art would readily be able to determine, without undue experimentation, the appropriate dosages (i.e., cell numbers, concentrations, vectors, etc.) to achieve the immune tolerance. When used in vivo for therapy, the formulations of the present invention are administered to the patient in therapeutically effective amounts; i.e., amounts that induce at least partial immune tolerance. As with all pharmaceuticals, the dose and dosage regimen will depend upon the nature of the antigen, the characteristics of the particular active agent (e.g., its therapeutic index), the patient, the patient's history and other factors. Again, dose and dosage regimen will vary depending on a number of factors known to those skilled in the art. See Remington's Pharmaceutical Science, 17th Ed. (1990) Mark Publishing Co., Easton, Pa.; and Goodman and Gilman's: The Pharmacological Basis of Therapeutics 8th Ed (1990) Pergamon Press.

The present invention also provides kits for carrying out the methods described herein. In one embodiment, the kit is made up of instructions for carrying out any of the methods described herein. The instructions can be provided in any intelligible form through a tangible medium, such as printed on paper, computer readable media, or the like. The present kits can also include one or more reagents, buffers, media, proteins, analytes, labels, antigens, genetic material encoding antigens, cells, such as engineered or non-engineered white blood cells, computer programs for analyzing results and/or disposable lab equipment, such as culture dishes or multi-well plates, in order to readily facilitate implementation of the present methods. Examples of preferred kit components can be found in the description above and in the following examples.

The present methods can involve any or all of the steps or conditions discussed above in various combinations, as desired. Accordingly, it will be readily apparent to the skilled artisan that in some of the disclosed methods certain steps can be deleted or additional steps performed without affecting the viability of the methods.

### EXAMPLE 1

In this example, the ability of AdαGT transduced lymphocytes to induce tolerance was determined in KO mice.

### Materials and Methods

Mice and Immunization Procedures

Inbred α1,3GT KO mice lacking α-gal epitopes [Thall et al. J Biol Chem 1995; 270: 21437-21442] on pure H-2b background, and their ‘syngeneic’ WT C57BL/6 counterpart, which differ only in that they also express α-gal epitopes, were used in this study. Experiments were performed with both males and females, and in compliance with the relevant laws and guidelines of the IACUC committee at Rush University, which approved the study. Activation of anti-Gal B cells and production of anti-Gal was achieved in KO mice by four weekly intraperitoneal immunizations, each with 50 mg PKM, as previously described. Ogawa, et al., Blood 2003; 101:2318, 2320; Mohiuddin, et al., Blood 2003; 102:229-236; Tanemura, et al., Transplantation 2002; 73:1859-1868; Tanemura, et al., J Clin Invest 2000; 105:301-310.

Transduction of KO Lymphocytes by AdαGT

The replication-defective adenovirus vector containing the α1,3GT gene (AdαGT) was propagated in 293 cells as previously described. Deriy et al. Glycobiology 2002; 12: 135-144. For transduction, KO lymphocytes were incubated for 4 h with AdαGT at ˜1×10¹⁰ infectious units/ml in RPMI medium containing 10% fetal bovine serum. Subsequently, the lymphocytes were washed and administered into mice via the tail vein, as 20×10⁶ lymphocytes/mouse. Control lymphocytes were transduced with the parental ‘empty’ adenovirus vector, [Gao et al. J Virol 1996; 70: 8934-8943] lacking α1,3GT insert.

Flow Cytometry Analysis of α-gal Epitope Expression on Transduced Lymphocytes

KO lymphocytes were incubated at a concentration of 10×10⁶ cells/ml for 30 min at 4° C. with 10 μg/ml fluoresceinated (FITC)-B. simplicifolia IB₄ lectin (BS lectin) (Sigma, St Louis, Mo., USA), in PBS containing 1% BSA. This lectin binds specifically to α-gal epitopes. (Wood, et al., Arch Biochem Biophys 1979; 198: 1-11). The cells were also stained with phycoerythrin (PE) labeled antimouse CD45R/B220 and anti-CD3 (Pharmingen, San Diego, Calif., USA) for identification of B and T cells, respectively. PE-conjugated anti-mouse CD11b/MAC-1 (Pharmingen) was used to stain monocytes, macrophages and dendritic cells. Cells were then washed, fixed and analyzed by FACSCalibur flow cytometer (Becton Dickinson, San Jose, Calif., USA).

PCR Analysis of Transduced Lymphocytes

Genomic DNA was extracted from spleen lymphocytes and subjected to PCR analysis for exon 9 of mouse α1,3GT gene (i.e. the exon containing the catalytic domain and which is disrupted in KO mice [Thall et al. J Biol Chem 1995; 270: 21437-2144]), using primers 5′-AGACTTTCTGGAGTCTGCTGACAT-3′ (SEQ ID NO: 1) and 5′-TACCTTGACACTTTTAATATCTGA-3′ (SEQ ID NO:2). (Larsen et al., Proc Natl Acad Sci USA 1989; 86:8227-8231). The PCR was performed with 35 cycles, each including 30 s at 94° C., 20 s at 62° C. and 20 s at 72° C., followed by 5 min at 72° C. The first cycle included 5 min at 94° C. to achieve complete denaturation of the DNA. The PCR yields a product of size 628 bp. Since this exon is disrupted in KO mice, a PCR product in this assay indicates the presence of the intact α1,3GT gene introduced by AdαGT, as previously demonstrated. (Deriy, et al., Glycobiology 2002; 12:135-144).

Tolerance Induction by AdαGT Transduced Lymphocytes

Tolerization of naive anti-Gal B cells. Naive KO mice received via the tail vein 20×10⁶ AdαGT transduced KO spleen lymphocytes on days 0, 4 and 9. The mice received four weekly PKM immunizations starting on day 14. Anti-Gal was determined a week after the fourth PKM immunization.

Tolerization of Memory Anti-Gal B Cells.

Spleen lymphocytes including memory anti-Gal B cells were obtained from KO mice immunized four times with PKM. (Mohiuddin et al., Blood 2003; 102:229-236; Tanemura, et al., J Clin Invest 2000; 105:301-310; Mohiuddin et al., Transplantation 2003; 75:248-262). Lymphocytes pooled from PKM-immunized mice were administered as 20×10⁶ cells/mouse into the tail vein of lethally irradiated KO mice (10.5 Gy). These mice also received 20×10⁶ bone marrow cells from naive KO mice for producing all needed blood series, and 20×10⁶ AdαGT transduced lymphocytes, or control 20×10⁶ KO lymphocytes transduced with the parental replication-defective adenovirus lacking the α1,3GT gene. Gao, et al., J Virol 1996; 70:8934-8943. Administration of transduced lymphocytes was repeated on days 4 and 9. PKM immunization was performed on days 14 and 21 post adoptive transfer and anti-Gal production determined on day 28.

ELISA Studies

Production of anti-Gal IgG in KO mice immunized with PKM was measured in serially diluted serum samples by ELISA in wells coated with 10 μg/ml synthetic α-gal epitopes coupled to BSA (α-gal-BSA, Dextra, Reading, UK). (Ogawa, et al, Blood 2003; 101: 2318-2320; Mihiuddin, et al, Blood 2003; 102:229-236; Tanemura et al., J Clin Invest 2000; 105:301-310; Mohiuddin et al., Transplantation 2003; 75:258-262). After 2 h incubation, the plates were washed and incubated with peroxidase-coupled goat anti-mouse IgG (Accurate Chemicals Labs, Westbury, N.Y., USA) and color developed with o-phenylenediamine (Sigma). Nonspecific binding was measured in ELISA wells coated with BSA and the data were subtracted from those in corresponding serum dilutions in ELISA with α-gal BSA. Anti-Gal IgM was measured by the same method using goat anti-mouse IgM (Accurate Chemicals Labs) as a secondary antibody. Since PKM-immunized mice produce many IgM antibodies that bind nonspecifically to ELISA wells, (Tanemura, et al., Transplantation 2002; 73:1859-1868) the sera were adsorbed on an equal volume of α-galactosidase-treated PKM (i.e. PKM lacking α-gal epitopes). This adsorption is performed for removal of such nonspecific IgM molecules prior to the ELISA for anti-Gal IgM.

An additional ELISA was performed for measuring production of IgG antibodies to the large variety of immunogenic pig xeno-proteins (anti-non-Gal antibodies). (Tanemura, et al., Transplantation 2002; 73:1859-1868). For this purpose, PKM (1 mg/ml) were dried in ELISA wells, resulting in their firm adherence. These membranes served as solid-phase antigen. Wells were blocked with 1% BSA in PBS. The assay was performed as that measuring anti-Gal above. The sera used were depleted of anti-Gal prior to the assay, by adsorption on glutaraldehyde-fixed rabbit red cells, which express an abundance of α-gal epitopes. (Galili et al., Proc Natl Acad Sci USA 1987; 84:1369-1373).

Heart Transplantation

Heterotopic transplantation of WT mouse hearts was performed as previously described. (Ogawa, et al., Blood 2003; 101:2318-2320; Mohiuddin et al., Blood 2003; 102:229-236; Mohiuddin et al., Transplantation 2003; 75:258-262). WT hearts from C57BL/6 donors are ‘syngeneic’ to KO mice but also express α-gal epitopes. KO mice were transplanted with these hearts in the abdominal cavity by connecting the WT pulmonary artery to vena cava inferior and the WT aorta to the KO aorta. At 4 weeks after transplantation, the mice were immunized with PKM every 2 weeks and tested for anti-Gal IgG response. The function of the heart was assessed by daily palpation.

### Results

Expression of α-gal Epitopes on Transduced KO Lymphocytes

Expression of α-gal epitopes on cells lacking it was achieved by the use of a replication-defective adenovirus vector, containing the ORF of the mouse α1,3GT gene. Deriy, et al., Glycobiology 2002; 12:135-144. This adenovirus vector, designated AdαGT, was found to transduce effectively human HeLa cells and induce the expression of ˜2×10⁶ α-gal epitopes/cell, within 24 h post transduction. Deriy, et al., Glycobiology 2002; 12:135-144. The efficacy of AdαGT transduction in inducing α-gal epitope expression on KO lymphocytes was analyzed at various days following administration of transduced lymphocytes into irradiated KO mice.

Expression of α-gal epitopes was determined by the binding of FITC-coupled Griffonia (Bandeiraea) simplicifolia IB4 lectin (BS lectin), which interacts specifically with α-gal epitopes (Wood, et al., Arch Biochem Biophys 1980; 198:1-11), and thus stains WT spleen lymphocytes (FIG. 3h) but not KO spleen lymphocytes (FIG. 3g). KO spleen lymphocytes obtained from the recipient of AdαGT transduced lymphocytes demonstrated expression of this epitope on 10-20% of spleen lymphocytes on days 1-3 post transduction (FIGS. 3b-d). However, this expression was only marginal on day 4 (FIG. 3e) and was not detected on day 7 (FIG. 3f). KO lymphocytes transduced with control ‘empty’ adenovirus vector (Gao, et al., J Virol 1996; 70:8934-8943) expressed no α-gal epitopes at any of the time points (FIG. 3a).

It was hypothesized that the observed diminished expression of α-gal epitopes on AdαGT transduced lymphocytes may be the result of destruction of the AdαGT genome by cellular endonucleases, within several days post transduction. To test this hypothesis, a PCR analysis for amplification of the intact exon 9 of α1,3GT gene was performed. This exon can be amplified from as little as 3 ng of WT lymphocyte DNA to yield a PCR product of 628 bp (FIG. 4a). In KO mice, exon 9 of the α1,3GT gene is disrupted by neomycin resistance gene (Thall et al. J Biol Chem 1995; 270: 21437-2144) and, therefore, is not amplified by PCR. Amplification of exon 9 occurs only if it is introduced into the cells in its intact form by the transducing AdαGT vector. The intact exon 9 was readily amplified from 100 ng DNA template of KO lymphocytes transduced with AdαGT, 24 h post transduction (FIG. 4b). Amplification of exon 9 within AdαGT transduced lymphocytes gradually decreased when the DNA template was obtained from lymphocytes on days 2 and 3, and could not be detected in DNA obtained from lymphocytes on day 4 post transduction (FIG. 4c). These mice were also studied for the presence of AdαGT in mesenteric and axillary lymph nodes and in the bone marrow. All PCR results were found to be negative for exon 9 when measured on day 4. This implied that the lack of spleen lymphocytes expressing α-gal epitopes on day 4 was indeed the result of the destruction of intracellular AdαGT and not because such cells migrated to other lymphoid organs.

Analysis of α-gal epitope expression in various lymphoid populations indicated that ˜23% of AdαGT transduced T cells (stained with anti-CD3), 15% of B cells (stained with anti-CD45R/B220) and 27% of monocytes, macrophages and dendritic cells (stained by anti-CD11b/Mac-1) expressed this epitope on day 2 post transduction (FIGS. 5b, e and h, respectively). All WT populations tested displayed positive shift for BS lectin staining (FIGS. 5c, f and i). In contrast, KO populations transduced with the parental adenovirus vector did not express α-gal epitopes (FIGS. 5a, d and g).

Tolerance Induction on Naive Anti-Gal B Cells by AdαGT Transduced KO Lymphocytes

The proportion of anti-Gal B cells in KO mice is too low for reliable identification by flow cytometry. Mohiuddin, et al., Blood 2003; 102:229-236; Tanemura, et al., J Clin Invest 2000; 105:301-310. Therefore, tolerization of these cells could not be monitored by flow cytometry, and had to be assessed by a functional assay measuring anti-Gal production following immunization with pig kidney membranes (PKM). Naive nonirradiated KO mice received 20×10⁶ AdαGT transduced KO lymphocytes via the tail vein. Since expression of α-gal epitopes on the transduced lymphocytes is transient and is limited to only few days (FIG. 3), administration of transduced lymphocytes was repeated on days 4 and 9. Anti-Gal production was measured by ELISA after four weekly PKM immunizations that started 14 days after the first administration of AdαGT transduced KO lymphocytes.

Control mice underwent a similar immunization protocol but received KO lymphocytes transduced with control parental adenovirus lacking the α1,3GT gene. (Gao, et al., J Virol 1996; 70-8934-8943). Whereas all 10 control mice displayed an effective anti-Gal IgG response, nine out of 10 mice receiving AdαGT transduced lymphocytes displayed no anti-Gal production, or only marginal activity (<1.0 OD at serum dilution of 1:50) of this antibody (FIG. 6a). The mean anti-Gal activity in the 10 tolerized mice was 32-fold lower than that in the control mice, that is, the mean of 0.5 OD observed in tolerized mice at serum dilution of 1:50 was observed in control mice in the 32-fold higher dilution of 1:1600 (FIG. 6b). This tolerance was specific to anti-Gal B cells, as demonstrated by ELISA studies with PKM as solid-phase antigen. Mice with tolerized anti-Gal B cells produced antibodies to pig peptide xenoantigens (antinon-Gal antibodies (Tanemura et al., Transplantation 2002; 73:1859-1968) in titers similar to those in control mice (FIG. 6c).

Tolerization of Memory Anti-Gal B Cells

Anti-Gal B cells in humans constitute as many as 1% of circulating B lymphocytes. Galili et al., Blood 1993; 82:2485-2493. Many of these B cells are lymphocytes primed by the cognate carbohydrate antigen on gastrointestinal bacteria. Galili et al., Infect Immun 1988; 56:1730-1737; Springer, et al., J Clin Invest 1969; 48:1280-1291. Memory B cells could be generated in KO mice by four PKM immunizations, in parallel to the production of anti-Gal. Tanemura et al., J Clin Invest 2000; 105:301-310. This elicited anti-Gal can destroy cells expressing α-gal epitopes; therefore, PKM-immunized KO mice are not suitable for studying tolerance induction by AdαGT transduced lymphocytes. Whereas anti-Gal can be effectively removed in primates by affinity columns expressing α-gal epitopes,(Galili, Springer Semin Immunopathol 1993; 15:155-171; Lin, et al., Transplantation 2000; 70:1667-1674; Watts et al., Xenotransplantation 2000; 7:181-185) such a treatment is technically not feasible in mice. Nevertheless, the detrimental effect of anti-Gal could be avoided in this experimental model by adoptive transfer of 20×10⁶ lymphocytes from PKM-immunized KO mice (i.e. lymphocytes including memory anti-Gal B cells) and 20×10⁶ naive KO bone marrow cells into lethally irradiated KO recipients. Mohiuddin, et al., Blood 2003; 102:229-236. Transferred memory anti-Gal B cells are less reactive when the recipient has an intact lymphoid system, than in lethally irradiated mice. Mohiuddin, et al., Blood 2003; 102:229-236. This is probably because of migration and function of transferred lymphocytes in lymphoid organs that are already ‘packed’ with autologous lymphocytes. Therefore, in order to measure the full potential of transferred memory anti-Gal B cells, the recipients were irradiated with 10.5 Gy. This eliminates autologous lymphocytes and allows for effective homing of the transferred lymphocytes including memory anti-Gal B cells into the recipient's lymphoid organs.

Each of the 18 mice in the control group and the 28 mice in the experimental group received 20×10⁶ lymphocytes including memory anti-Gal B cells from the same pool of lymphocytes obtained from PKM immunized KO mice and 20×10⁶ bone marrow cells from naive KO mice (to provide for other blood cell series). Control mice also received KO lymphocytes transduced with the parental ‘empty’ adenovirus vector whereas experimental mice received KO lymphocytes transduced with AdαGT. All control mice effectively produced anti-Gal IgG after two PKM immunizations (closed circles in FIGS. 7a and b). In contrast, 57% of mice in the experimental group also receiving AdαGT transduced lymphocytes (16 out of 28 mice represented by open circles) displayed no anti-Gal IgG response or very low production of this antibody (<1.0 OD at serum dilution of 1:50). The mean anti-Gal IgG response in these 16 mice was >64-fold lower than that in the control mice receiving the same memory anti-Gal B cells (FIG. 7b). No increase in anti-Gal IgG response was observed in the 16 mice after two additional PKM immunizations on days 28 and 35. Overall, the findings in these 16 mice suggest an effective induction of tolerance on memory anti-Gal B cells by AdαGT transduced lymphocytes. The remaining 12 mice in the experimental group (triangles in FIGS. 7a and b) displayed a low anti-Gal response (>1.0 OD at serum dilution of 1:50 in FIG. 7a), which on average was 32-fold lower than that in the control group (FIG. 7b). These findings suggest that tolerance induction in this group may not have been fully achieved.

The mice in the tolerized group and in the control group in FIGS. 7a and 7b were further studied for anti-Gal IgM response. As shown in FIG. 7c, control mice displayed effective production of anti-Gal IgM whereas tolerized mice failed to exhibit significant levels of this anti-Gal isotype.

Transplantation of WT Heart Expressing α-gal Epitopes into Tolerized Mice

The suggestion that memory anti-Gal B cells are tolerized by AdαGT transduced KO lymphocytes was further supported by studies on transplantation of ‘syngeneic’ mouse heart expressing α-gal epitopes (i.e. WT heart from C57BL/6 mice). The timeline for this study is described in Table 1. It was previously reported that transplantation of WT hearts into KO mice producing anti-Gal results in hyperacute rejection because of anti-Gal binding to α-gal epitopes on the WT endothelial cells of the transplanted heart. Ogawa et al., Blood 2003; 101:2318-2320; Mohiuddin, et al., Blood 2003; 102:229-236. Accordingly, eight of the control mice in FIGS. 7a and b, which were heterotopically transplanted with WT mouse heart on day 28, rejected these hearts within 30 min to 18 h (Table 1). The previous studies indicated that this rejection is associated with deposits of anti-Gal IgM and IgG on the blood vessel walls and that the binding of this antibody to the α-gal epitopes on WT cells results in complement-mediated cytolysis of these cells. Ogawa et al., Blood 2003; 101:2318-2320; Mohiuddin, et al., Blood 2003; 102:229-236; Mohiuddin et al., Transplantation 2003; 75:248-262.

TABLE 1
Perpetuation of tolerance to α-gal epitopes by transplantation of WT heart

| Irradiation and adoptive | | | | | | | | | | |
|---|---|---|---|---|---|---|---|---|---|---|
| transfer of AdαGT | | | | | | | | | | |
| transduced lymphocytes | Administration of | | | | Heterotopic | | | | | Removal of |
| and of lymphocytes | AdαGT transduced | | | | WT heart | PKM | | | | transplanted |
| from PKM immunized mice | lymphocytes | | PKM Immunizations | | transplantation | Immunizations | | | | WT heart |
| ↓ | ↓ | ↓ | ↓ | ↓ | ↓ | ↓ | ↓ | ↓ | ↓ | ↓ |
| Time: 0 | Days 4 | 9 | Days 14 | 21 | Day 28 | Weeks 8 | 10 | 12 | 14 | Week 19 |

| | | Additional PKM | |
|---|---|---|---|
| | | immunizations | |
| Group | Number of mice | post transplantation | Survival time of WT heart |
| Tolerized^{a} | 2 | 2 | 45 days^{c} |
| Tolerized^{a} | 3 | 3 | 62-64 days^{d} |
| Tolerized^{a} | 4 | 4 | 100 days^{c} |
| Control^{b} | 8 | N/A | 0.5-18 h |

^{a}No anti-GaI IgG response post repeated PKM immunizations was detected in any of the tolerized mice.
^{b}All control mice produced anti-GaI IgG in high titers.
^{c}Hearts were removed for histological evaluation.
^{d}Mice died of unknown reason.

A similar procedure of heterotopic transplantation of syngeneic WT heart was performed on day 28 in nine of the tolerized mice presented in FIG. 7a. No transplanted WT hearts were rejected in tolerized KO mice, as evaluated by daily palpation (Table 1). Starting 4 weeks after WT heart transplantation, the mice were repeatedly immunized with PKM every 2 weeks. The transplanted WT hearts continued to function despite these repeated immunizations (Table 1). Two of the transplanted WT hearts that were removed on day 45 displayed normal histologic characteristics (not shown). Three of the mice died 62-64 days post WT heart transplantation of unknown reasons, but the WT hearts in these mice were not rejected prior to death. In the remaining four mice, transplanted WT hearts continued to function for 100 days despite four PKM immunizations given after these hearts were transplanted (Table 1). Accordingly, no anti-Gal production was observed in these mice, 1 week after the fourth PKM immunization (FIG. 8). In contrast, control mice, receiving memory anti-Gal B cells from the same pool of lymphocytes as the tolerized mice, but which were not transplanted with WT heart, readily produced anti-Gal following such PKM immunizations (FIG. 8). These findings suggest that tolerance induced by AdαGT transduced lymphocytes is perpetuated for long periods in mice transplanted with WT heart.

Analysis of Deletion or Anergy of Memory Anti-Gal B Cells in Tolerized Mice

Previous studies have shown that tolerance to various protein antigens can be induced either by deletion or by anergy of the B cells with the corresponding specificities. Goodnow et al., Nature 1988; 334:676-682; Goodnow et al., Nature 1991; 352:532-536; Nemazee et al., Nature 1989; 337:562-566. Since the proportion of physiologic memory anti-Gal B cells is low, (Mohiuddin et al., Blood 2003; 102:229-236; Tanemura et al., J Clin Invest 2000; 105:301-310) the fate of these cells could not be accurately determined by flow cytometry analysis, as performed in transgenic mice. Goodnow et al., Nature 1988; 334:676-682; Goodnow et al., Nature 1991; 352:532-536; Nemazee et al., Nature 1989; 337:562-566. Therefore, deletion or anergy of memory anti-Gal B cells was studied by a functional assay determining their ability to produce anti-Gal after secondary adoptive transfer. The spleens in the tolerized mice (open circles in FIG. 7a) contained ˜0.5×10⁸ lymphocytes per spleen. These lymphocytes were washed and transferred into irradiated KO mice as 20×10⁶ cells per mouse, 1 week after the second PKM immunization (i.e. on day 28). In accord with the data in FIGS. 3 and 4c, these transferred lymphocytes lacked cells expressing α-gal epitopes and yielded no PCR product of exon 9 of the transduced α1,3GT gene even with 500 ng DNA as template (FIG. 4d). This implied that AdαGT was absent in lymphocytes that were transferred from the tolerized mice into secondary recipients.

Five secondary recipients of lymphocytes, each from a different tolerized mouse, were immunized with PKM, 14 and 21 days post adoptive transfer. These recipients displayed no significant anti-Gal IgG response when measured on day 28 post secondary adoptive transfer (FIG. 9a). In contrast, secondary recipients of lymphocytes from the control nontolerized mice of FIG. 7a displayed an effective anti-Gal response after two PKM immunizations (FIG. 9a). The effective production of anti-Gal in the latter group implies that memory anti-Gal B cells in control nontolerized mice maintained their activity and were not affected by the secondary adoptive transfer.

Previous studies indicated that anergized B cells revert into an active state within 10 days after the removal of the anergizing antigen. Goodnow et al., Nature 1991; 352:532-536. In the present study, lymphocytes transferred from tolerized mice resided in secondary KO recipients for 14 days in the absence of α-gal epitopes. Based on the studies in Goodnow et al., (Nature 1991; 352:532-536) this period of 14 days in the secondary recipient should have sufficed for the reversion of anergized memory anti-Gal B cells into competent B cells that are activated as a result of PKM immunization. Thus, the lack of significant anti-Gal response in the secondary recipients suggests that memory anti-Gal B cells are physically absent, that is, they could have been deleted upon encountering α-gal epitopes on AdαGT transduced lymphocytes.

The process of tolerance induction by AdαGT transduced lymphocytes was further studied for the possible association with activity of regulatory lymphocytes, since such cells were found to control the immune response in a number of experimental models. Sakaguchi et al., Immunol Rev 2001; 182:18-32; Shevach, Nat Rev Immunol 2002; 2:389-400. To study the possible presence of regulatory lymphocytes that may downregulate activity of anti-Gal B cells in tolerized mice, the secondary adoptive transfer experiments were repeated with mixed populations of lymphocytes from tolerized mice and from PKM-immunized control mice. The secondary recipients received two PKM immunizations, starting 14 days post adoptive transfer. It was assumed that if regulatory T cells are present in the tolerized mice, then such cells should downregulate the activity of memory anti-Gal B cells that originated in the control mice. The recipients of lymphocyte populations from both tolerant and control mice produced anti-Gal in titers that were only slightly lower and not significantly different from those in recipients of lymphocytes from only control mice (FIG. 9b). This suggests that even if there are regulatory T cells in tolerized mice, these cells cannot account for the effective prevention of anti-Gal response in tolerized mice of FIG. 7a.

Discussion of Results

The present study demonstrates induction of tolerance on naive and memory anti-Gal B cells by administration of KO lymphocytes manipulated to express α-gal epitopes, following in vitro transduction of such lymphocytes with AdαGT. Studies of secondary transfer of lymphocytes from tolerized KO mice to irradiated recipients suggest that the observed tolerance is the outcome of deletion of anti-Gal B cells that engage α-gal epitopes on AdαGT transduced lymphocytes. Since the α-gal epitope by itself lacks the ability of activating T cells, (Tanemura et al., J Clin Invest 2000; 105:301-310) it is possible that the observed tolerance is the result of anti-Gal B cells engaging cell-surface α-gal epitopes in the absence of T-cell help. It was previously proposed the occurrence of such a tolerizing mechanism in KO mouse recipients of ‘syngeneic’ WT lymphocytes. (Mohiuddin et al., Blood 2003; 102:229-236). The studied anti-Gal B cells are physiologic B cells, rather than transgenic lymphocytes. Therefore, it is impossible at present to determine the exact mechanism for the B cell elimination. This tolerance could be the result of physical deletion of these B cell or may result from changes in antigenic specificity of anti-Gal B cells following receptor editing of the immunoglobulin genes in these B cells. (Radic et al., J Exp Med 1993; 177: 1165-1173; Tiegs et al., J Exp Med 1993; 177: 1009-1020). In addition, the possibility that the anti-Gal B cells are anergized for periods longer than 14 days cannot be completely excluded at present, although previous studies indicated that <10 days are required for reversion of anergized B cells into an active state.(Goodnow et al., Nature 1991; 352:532-536). Only studies in ‘knock in’ transgenic mice producing anti-Gal, that is, mice in which a large proportion of B cells produce anti-Gal as the product of targeted transgene inserted into the immunoglobulin gene region, will enable an accurate characterization of the mechanism for tolerance induction in this experimental model.

Not all mice were fully tolerized, as some exhibited partial production of anti-Gal (˜32-fold less than control mice). Previous studies on tolerance induction by WT lymphocytes indicated that the tolerizing WT lymphocytes and anti-Gal B cells ‘need time to find each other’ in order for the B cells to be tolerized. (Mohiuddin et al., Blood 2003; 102:229-236). Anti-Gal B cells that are not tolerized by the time of PKM immunization (i.e. within the period of 14 days) may be rescued from tolerance by T-cell help, resulting from the activation of helper T cells by the multiple immunogenic pig xenopeptides. (Mohiuddin et al., Blood 2003; 102:229-236). Activity of such rescued anti-Gal B cells may explain the low anti-Gal activity in 12 of the 28 mice treated for tolerization of memory anti-Gal B cells. It is possible that repeated administration of AdαGT transduced lymphocytes for >14 days may increase the proportion of mice that are effectively tolerized to the α-gal epitope.

Although the expression of α-gal epitopes on AdαGT transduced lymphocytes lasts for 3-4 days, the tolerance induced by these lymphocytes can be perpetuated for prolonged periods by the subsequent transplantation of WT heart expressing this epitope. The transplanted WT hearts continued to function in the tolerized mice for 100 days, and these mice did not produce anti-Gal despite additional PKM immunizations post transplantation (Table 1 and FIG. 8). It is probable that newly formed anti-Gal B cells that emerge in the bone marrow in these transplanted mice ‘regard’ the α-gal epitope on the graft as a self-antigen and thus are tolerized by it.

Previous studies in this experimental animal model demonstrated induction of tolerance to α-gal epitopes by administration of syngeneic WT bone marrow cells expressing α-gal epitopes (Yang et al., J Exp Med 1998; 187:1335-1342; Ohdan et al, J Clin Invest 1999; 104:281-290) or by administration of autologous bone marrow cells transfected with retrovirus containing the α1,3GT gene. Bracy et al., Science 1998; 281-1845-1947; Bracy et al., Blood 2000; 96:3008-3015. Previous studies (Ogawa et al., Blood 2003; 101:2318-2320; Mohiuddin et al., Blood 2003; 102:229-236) and the present study, all indicate that this tolerance can be induced also by cells expressing α-gal epitopes, other than bone marrow cells. Since all KO mice are syngeneic, the administered AdαGT transduced KO lymphocytes may be regarded as autologous lymphocytes inducing tolerance. The present example further supports the possibility that tolerance to α-gal epitopes may be induced by a similar method in humans. Lymphocytes obtained from the blood and transduced in vitro with AdαGT may tolerize naive and memory anti-Gal B cells following their administration back into the patient. Tolerization of anti-Gal B cells by autologous lymphocytes expressing α-gal epitope can include the removal of the natural anti-Gal from the circulation, in order to prevent destruction of the tolerizing lymphocytes by the antibody. This can be achieved by affinity column expressing synthetic α-gal epitopes. Galili, Springer Semin Immunopathol. 1993; 15-155-171; Lin et al, Transplantation 2000; 70:1667-1674; Watts et al., Xenotransplantation 2000; 7:181-185. Since the transduction of lymphocytes by AdαGT will be performed in vitro, it is probable that this method of gene therapy will not be affected by factors limiting in vivo gene therapy by adenovirus vectors, such as immune response to the virus. Chirmule et al., Gene Therapy 1999; 6:1574-1583.

The relevance of this method for induction of tolerance to α-gal epitopes in humans will first have to be tested in monkeys in order to determine whether this phenomenon, which is observed in mice, is also applicable to primates. If tolerance induction by autologous AdαGT transduced lymphocytes is observed in monkeys, similar induction of tolerance may be considered for incompatible blood group A (GalNAcα1-3[α1-2Fuc]-Gal β1-4GlcNAc-R) or B antigens (Galα1-3[α1-2Fuc]-Gal α1-4GlcNAc-R). The structure of these cell-surface carbohydrate antigens is very similar to that of the α-gal epitope (Galα1-3Galβ1-4GlcNAc-R). Moreover, a large proportion of anti-blood group antibodies are in fact anti-Gal antibodies that bind to the α-gal epitope that serves as the core structure for these blood group antigens. Galili et al., J Exp Med 1987:165: 693-704; Galili, Transfus Med Rev 1988; 2:112-121; Galili et al., Transplantation 2002; 74:1574-1580. ABO incompatibility is presently one of the major limiting factors in transplantation of allografts in humans. Starzl et al., Surgery 1964; 55:195-200; Wilbrandt et al., Am J Clin Pathol 1969; 51:15-23; Rydberg., Transfus Med 2001; 11:325-342; Cooper., J Heart Transplant 1990; 9:376-381. By using autologous lymphocytes transduced with adenovirus containing A or B transferase gene or both, (Yamamoto et al., Nature 1990: 345:229-223), one may induce tolerance similar to that observed with AdαGT transduced lymphocytes. As with tolerance induction to the α-gal epitope, such a treatment can also include depletion of the corresponding anti-blood group antibody, prior to the administration of the transduced lymphocytes. If successful, this method may ultimately allow for the transplantation from ABO-incompatible living donors without the risk of rejection as a result of immune response to the incompatible blood group antigen, such as of kidney allografts and ABO-incompatible heart in patients requiring urgent heart transplantation.

The use of the article “a” or “an” is intended to include one or more.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

All references disclosed herein are specifically incorporated by reference thereto. While preferred embodiments have been illustrated and described, it should be understood that changes and modifications can be made therein in accordance with ordinary skill in the art without departing from the invention in its broader aspects as defined in the following claims.

The following references are hereby incorporated into the patent application in their entirety:

1. Bensinger W I, Baker D A, Buckner C D, Clift R A, Thomas E D. Immunoadsorption for removal of A and B blood-group antibodies. N Engl. J. Med. 304: 160, 1981.

2. Deriy L, Chen Z-C, Gao G-P, Galili U. Expression of α-gal epitopes on HeLa cells transduced with adenovirus containing a 1,3galactosyltransferase cDNA. Glycobiology 2002; 12: 135-144.

3. Galili U. Evolution and pathophysiology of the human natural anti-α-galactosyl IgG (anti-Gal) antibody. Springer Semin. Immunopathol. 15: 155, 1993.

4. Galili U. Interaction of the natural anti-Gal antibody with α-galactosyl epitopes: A major obstacle for xenotransplantation in humans. Immunology Today 14:480, 1993.

5. Galili U, Rachmilewitz E A, Peleg A, Flechner I. A unique natural human IgG antibody with anti-α-galactosyl specificity. J. Exp. Med. 160:1519, 1984.

6. Galili U, Macher B A, Buehler J, Shohet S B. Human natural anti-α-galactosyl IgG. II. The specific recognition of α(1-3)-linked galactose residues. J. Exp. Med. 162:573, 1985.

7. Galili U, Shohet S B, Kobrin E, Stults C L M, Macher B A. Man, apes, and Old World monkeys differ from other mammals in the expression of α-galactosyl epitopes on nucleated cells. J. Biol. Chem. 263:17755, 1988.

8. Galili, U., R. E. Mandrell, R. M. Hamahdeh, S. B. Shohet and J. M. Griffis. Interaction between human natural anti-α-galactosyl immunoglobulin G and bacteria of the human flora. Infect. Immun. 1730-1737, 1988.

9. Galili U, LaTemple D C, Radic M Z. A sensitive assay for measuring α-Gal epitope expression on cells by a monoclonal anti-Gal antibody. Transplantation 65: 1129, 1998.

10. Gao G P, Yang Y, Wilson J M. Biology of adenovirus vectors with E1 and E4 deletions for liver-directed gene therapy. J. Virol. 70: 8934, 1996.

11. Larsen R D, Rajan V P, Ruff M M, Kukowska-Latallo J, Cummings R D, Lowe J B. Isolation of a cDNA encoding a murine UDPgalactose:β-D-galactosyl-1,4-N-acetyl-D-glucosaminide α-1,3-galactosyltransferase: expression cloning by gene transfer. Proc Natl Acad Sci USA 86: 8227, 1989.

12. Misrahi M, Loosfelt H, Atger M, Sar S, Guiochon-Mantel A, Milgrom E. Cloning, sequencing and expression of human TSH receptor. Biochem Biophys Res Commun. 166: 394, 1990.

13. Mohiuddin M M, Ogawa H, Yin D-P, Galili U. Tolerance induction to a mammalian blood group like carbohydrate antigen by syngeneic lymphocytes expressing the antigen. II. Tolerance induction on memory B cells. Blood 2003; 102: 229-236.

14. Ogawa H, Yin D-P, Shen J, Galili U. Tolerance induction to a mammalian blood group like carbohydrate antigen by syngeneic lymphocytes expressing the antigen. Blood 2003; 101:2318-2320.

15. Ogawa H, Yin D-P, Galili U. Induction of immune tolerance to a transplantation carbohydrate antigen by gene therapy with autologous lymphocytes transduced with adenovirus containing the corresponding glycosyltransferase gene. Gene Therapy 11: 292, 2004.

16. Speir J A, Abdel-Motal U M, Jondal M, Wilson I A. Crystal structure of an MHC class I presented glycopeptide that generates carbohydrate-specific CTL. Immunity 10: 51, 1999.

17. Tanemura M, Yin D, Chong A S, Galili U.: Differential immune responses to α-gal epitopes on xenografts and allografts: implications for accommodation in xenotransplantation. J. Clin. Invest. 105: 301, 2000.

18. Yamamoto F, Clausen H, White T, Marken J, Hakomori S. Molecular genetic basis of the histo-blood group ABO system. Nature 345: 229, 1990.

19. Wood C, Kabat E A, Murphy L A, Goldstein I J. Immunochemical studies of the combining sites of the two isolectins, A4 and B4, isolated from Bandeiraea simplicifolia. Arch Biochem Biophys 1979 198: 1-11.

What is claimed is:

1. A method of inducing immune tolerance to an antigen in a mammal, comprising:
(a) administering an engineered population of white blood cells that express an antigen to a mammal one or more times thereby inducing at least partial immune tolerance of the antigen in the mammal.

2. The method of claim 1 further comprising:
(b) engineering a population of white blood cells to express the antigen.

3. The method of claim 2 further comprising:
(c) obtaining the population of white blood cells from the individual prior to (b).

4. The method of claim 2 wherein (b) comprises inserting a nucleic acid encoding the portion of the antigen or a nucleic acid that encodes an enzyme capable of producing part of the antigen into the white blood cells.

5. The method of claim 4 wherein the nucleic acid encoding the portion of the antigen or a nucleic acid that encodes an enzyme capable of producing part of the antigen is inserted into the white blood cells by a replication defective adenovirus.

6. The method of claim 1 wherein the antigen is a carbohydrate.

7. The method of claim 6 wherein the antigen is a blood group antigen.

8. The method of claim 7 wherein the blood group antigen is blood group A antigen, blood group B antigen or both.

9. The method of claim 2 wherein (b) occurs in vitro.

10. A white blood cell produced by engineering the white blood cell to express an antigen.

11. A pharmaceutical composition comprising the cell of claim 10.

12. The method of claim 1 further comprising:
(d) exposing the mammal to the antigen.

13. The method of claim 11 wherein (d) comprises transplanting a tissue comprising the antigen into the mammal.

14. The method of claim 1 wherein the mammal is a human.

15. The method of claim 12 further comprising:
(e) measuring the immune reaction of the mammal to the antigen.

16. The method of claim 15 further comprising:
(f) comparing the immune reaction of the mammal to the antigen with the immune reaction of a control mammal that had not been administered an engineered population of white blood cells that express the antigen.

17. The method of claim 6 wherein the antigen comprises the α-gal epitope [Galα1-3Galβ1-(3)4GlcNAc-R].

18. The method of claim 1 wherein the mammal is essentially free of circulating antibodies that react specifically with the antigen.

19. The method of claim 1 wherein the engineered white blood cells comprise lymphocytes.
